(19) United States
(12) Patent Application Publication
(10) Pub. No.: US 2019/0053604 A1
(43) Pub. Date: Feb. 21, 2019

(54) DEVICE FOR PACKAGING AN EMULSION

(71) Applicant: CAPSUM, Marseille (FR)

(72) Inventor: Sèbastien BARDON, Paris (FR)

(21) Appl. No.: 15/763,678

(22) PCT Filed: Sep. 28, 2016
(86) PCT No.: PCT/EP2016/073079

§ 371 Date: Mar. 27, 2018

(30) Foreign Application Priority Data
Sep. 28, 2015 (FR) 1559134

(51) Int. Cl. A45D 40/22; A45D 34/04; A45D 34/00; A45D 40/18; A61K 8/06; A61Q 19/00; A61Q 1/00; A61K 8/898; A61K 8/81; A61K 8/34

(52) U.S. Cl. CPC A45D 40/22; A45D 34/042; A45D 34/04; A45D 34/00; A45D 40/18; A61K 8/062; A45D 42/00; A61Q 1/00; A61K 8/898; A61K 8/8152; A61K 8/345; A45D 2200/1018; A61Q 19/00

(57) ABSTRACT

Disclosed is a packaging device comprising at least: i) a receptacle having at least one reservoir containing at least one emulsion with a continuous phase and a dispersed phase in the form of drops, wherein the drops comprise a shell with at least one cationic polymer and at least one anionic polymer; ii) an applicator for applying the emulsion to a keratin material; and iii) an element having at least one orifice allowing the emulsion to pass from the reservoir to the applicator.

The present invention relates to a packaging device comprising at least one emulsion, or even a cosmetic composition, in particular perfumed.

The present invention also relates to a non-therapeutic cosmetic process for makeup and/or care of a keratin material and/or for improving perfume retention on a keratin material.

There are many types of compositions in the cosmetic field, especially in the form of emulsions, that are, in particular, stabilized by surfactants. There are also emulsions which may comprise a perfuming agent and which are stable despite a high perfume concentration and the absence of surfactants (FR 2 973 356). These types of compositions are commonly adapted for topical application with the fingers of the user's hand, or may be formulated as a spray.

However, consumers are constantly looking for cosmetics that are easy to use and apply, and which also give them good sensory properties.

In addition, the provision of new cosmetic products to consumers, in particular by means of unconventional methods of application for a desired cosmetic purpose (sensory properties, texture, etc.), remains a constant objective in this field.

The present invention aims to meet these needs.

The present invention therefore aims to provide a new packaging device for simple and fast application, while providing cosmetics with interesting or improved sensory properties for the user.

Another object of the present invention is to provide a device that is easy to carry and/or use.

The present invention therefore relates to a packaging device comprising:
i) a receptacle comprising at least one reservoir containing at least one emulsion comprising a continuous phase and a dispersed phase in the form of drops, wherein the drops comprise a shell formed by at least one cationic polymer and at least one anionic polymer;
ii) means for applying the emulsion to a keratin material; and
iii) an element having (or comprising) at least one orifice allowing the passage of the emulsion from the reservoir to the application means.

The emulsion according to the invention may preferably comprise at least one perfuming agent, as described in more detail hereinafter, and, in particular, may comprise at least 2% by weight, in particular at least 3% by weight, preferably 3% to 35% by weight, in particular from 5% to 20% by weight, better still from 5% to 15% by weight, and even more preferably from 5% to 10% by weight, of perfuming agent(s) relative to the total weight of the emulsion.

In the context of the invention, and unless otherwise stated, the term “packaging device” refers to any packaging enabling the packaging, sale, transport, protection and storage of the cosmetic product it contains. More particularly, within the meaning of the present invention, the packaging device extends to the cosmetic product within the packaging and intended directly for the end user.

According to one embodiment, the packaging device may also be an application device, in particular for the application of the emulsion, on a keratin material.

Packaging Device

Receptacle

According to the invention, the reservoir (or chamber) may comprise a volume of from 5 ml to 200 ml, preferably from 10 ml to 100 ml, and in particular from 15 ml to 50 ml.

According to the invention, the element comprising at least one orifice is chosen from the group consisting of a sponge, a rigid plate pierced with at least one hole, preferably pierced with a plurality of holes, or a net or a sieve.

According to one embodiment, the element having at least one orifice is a sponge, in particular a cellular sponge. This type of sponge is generally called an “air cushion” in the cosmetic field.

Typically, when the element having at least one orifice is a sponge, it is soaked (or impregnated) with the emulsion according to the invention. In other words, the emulsion is inside the sponge.

According to one embodiment, the element having at least one orifice, when represented by a “rigid plate pierced with hole(s)” is a grid. In this case, the orifice of the grid corresponds to a hole.

According to one embodiment, when the element having at least one orifice is a sieve or a net, it consists of a crosswork of synthetic son forming a mesh.

The mesh size of the sieve may vary from 5 μm to 600 μm, preferably from 30 μm to 200 μm.

The use of sieves or grids advantageously allows the emulsion to pass through the interstices between the mesh of the sieve or between the holes of the grid.

The viscosity of the emulsion according to the invention is typically adapted to the nature of the element having at least one orifice. More particularly, the viscosity of the emulsion according to the invention is designed to ensure a satisfactory and desired passage of the emulsion through the orifice(s), in particular upon bringing the element having at least one orifice into contact with the application means by the user and application of pressure by the latter on the element.

According to one embodiment, the element having at least one orifice is therefore designed to allow the passage of a quantity of the emulsion, and thus to coat, at least partially, the application means in response to the pressure exerted by the user on the element via the application means. Thus, the packaging device according to the invention can absorb the right dose of the emulsion upon pressing the application means against the element having at least one orifice. The emulsion according to the invention may thus become multi-functional because the application is then personalized by using the application means; in fact, the application of the emulsion little by little makes it possible to easily adjust the amount of product applied, and thus the coverage and/or loading of perfuming agent(s) from the most subtle to the most intense.

According to the invention, the receptacle may be rechargeable, i.e. it is possible to recharge the emulsion in the reservoir, especially when the reservoir becomes empty after several uses.

According to one embodiment, the element comprising at least one orifice is removable.

According to the invention, when the element having at least one orifice is a sponge, it may be refilled by replacing the sponge.

The rechargeable nature of a device according to the invention is advantageous because it is ecological and also allows the hue to be varied depending on the season.

According to one embodiment, the element comprising at least one orifice allows the closure of one of the reservoir surfaces, in particular the surface opposite the bottom of the reservoir.

According to one embodiment, when the element comprising at least one orifice is a sieve or a grid, the element makes it possible to close the reservoir, in particular to close the receptacle.

According to one embodiment, when the element comprising at least one orifice is a sponge, the element is comprised inside the reservoir, in particular in the receptacle.

According to this embodiment, the device may further comprise at least one additional element capable of closing the reservoir, and in particular the receptacle.

According to one embodiment, when the element comprising at least one orifice is a sponge, it may comprise the emulsion according to the invention.

According to one embodiment, upon using the device, the user is faced with the element having at least one orifice when the device is open.

According to one embodiment, when the element comprising at least one orifice is a rigid plate pierced with at least one hole, a sieve or a net, the device according to the invention may also comprise a device for pumping the emulsion.

According to the invention, the term “pumping device” or “suction device”, refers to a device for pumping or sucking the emulsion present in the receptacle, and more particularly present in the reservoir, towards the application means.

According to one embodiment, the pumping device is located in the receptacle, and more particularly between the element comprising at least one orifice and the reservoir of the receptacle, and, in particular, between the element comprising at least one orifice and the emulsion according to the invention.

According to one embodiment, the element comprising at least one orifice and the pumping device, are integral.

The pumping device advantageously allows the passage of the emulsion from the reservoir towards the applicator, in particular by passing through the orifice(s) of the element having at least one orifice, especially when the user is exerting a pressure on the element comprising at least one orifice.

According to one embodiment, the receptacle consists of the reservoir containing the emulsion and the element comprising at least one orifice.

According to one embodiment, the receptacle consists of the reservoir containing the composition, the element comprising at least one orifice and the pumping device.

Application Means

In the context of the invention, and unless otherwise indicated, the term “application means” or “applicator element” or “application member” or “applicator” refers to an element that makes it possible to take up at least a part of the emulsion comprised in the reservoir and to apply it to a surface, in particular to a keratin material.

According to one embodiment, the application means is selected from the group consisting of a brush, a puff, a pad, a foam, a stick, for example a silicone stick, and a mini-brush. The applicator may have any shape, including any cross-section. For example, the applicator may have a circular, oval, or polygonal cross-section, for example square or triangular.

According to one embodiment, the applicator is a foam, in particular in the form of a disc.

In the present invention, the foam may also be referred to as a “cushion” or a “pad”. According to the invention, the foam may be synthetic.

According to one embodiment, the applicator is made of foam, which may or may not be crosslinked, and which may comprise open or closed cells. Preferably, the applicator comprises a crosslinked polyurethane foam layer.

The foam may be selected from the group consisting of polyvinyl chloride (PVC), polyurethane, polyether, polyester; an elastomer of SBR (Synthetic Butadiene Rubber), NBR, silicone, nitrile type, and their mixtures.

In another embodiment, the applicator may be a closed-cell non-cross-linked Rubycell foam having a pore diameter of from 50 to 500 pores per inch. Rubycell is, in particular, marketed by Toyo Polymer Co. Ltd.

Preferably, the foam is a mixture of polyurethane and Rubycell.

According to the invention, the applicator may be a foam consisting of one of the following flocked materials: nylon, polyesters, acrylics, cotton, and mixtures thereof.

According to one embodiment, the applicator is a foam consisting of one of the following materials: polyester, polyvinyl chloride (PVC), PVA (polyvinyl alcohol), polyurethane, polyether, SBR (Synthetic Butadiene Rubber) type elastomer, NBR (nitrile butadiene rubber), silicone or nitrile type, natural or synthetic rubber (especially with closed cells), polyolefin, EDPM (Ethylene, Propylene, Diene, Monomer), cellulose, or mixtures thereof.

According to one embodiment, the applicator is a foam called Yukilon, marketed by Yukigaya Chemical Industries Co., Ltd., Tokyo, Japan.

According to one embodiment, the applicator comprises one or more layers of polyethylene, polyurethane or polypropolene, synthetic or natural latex, EDPM, rubber, styrene, nylon, coated paper, polyolefin, plastics made of nylons or polyesters, Teflon, PVA or PVC.

According to one embodiment, the applicator is made of cotton, in particular woven cotton, woven nylon, acrylic, wool, flannel, polyester, cotton, or their mixtures.

According to one embodiment, the applicator is made of knitted fabric of cotton, polyester, acrylic, nylon, flannel, felt, or their mixtures.

According to one embodiment, the foam is microporous, and, in particular, comprises pores with a diameter of between 10 and 500 μm.

According to one embodiment, the applicator may be independent of any support, particularly when it is a foam.

According to one embodiment, the applicator is secured to a support advantageously allowing the user to grip the applicator without contact therewith. This advantageously avoids the user having direct contact with the emulsion to be applied, and thus soiling the fingers with the emulsion to be applied.

According to one embodiment, upon exerting a pressure of the applicator on the element having at least one orifice of the receptacle, the applicator is at least partially compressed and the element having at least one orifice then deforms thus reducing the available volume for the emulsion in the reservoir, so that at least a portion of the emulsion may exit through the orifice and thus come into contact with the surface of the applicator. Thus, the user may take up a certain dose of the emulsion on the applicator and then deposit it on the keratin material.

According to one embodiment, when the packaging device according to the invention comprises a pumping device as described above, upon exerting a pressure of the applicator on the element having at least one orifice of the receptacle, a part of the emulsion may pass from the reservoir to the application means via the pumping device.

Typically, the pressure exerted by the user may be a pressure performed by tamping the applicator on the element having at least one orifice.

According to the invention, the applicator may be housed above the reservoir, or even the receptacle, when the device is closed, and therefore not in use.

Device

The packaging device according to the invention may comprise at least one closure member that is intended to close the receptacle, preferably in a sealed manner when not in use.

According to one embodiment, the applicator is housed on one surface of the closure member. Advantageously, the closure member makes it possible to separate the applicator from the receptacle comprising the emulsion, which makes it possible to avoid contaminating the applicator when the device is not in use.

The device may comprise a housing provided with a lid and a body. Preferably, the housing has a cylindrical shape.

Preferably, the device is a powder compact.

The device may also comprise a mirror arranged on the inner surface of the lid.

Preferably, the mirror covers the entire inner surface of the cover in order to provide the most comfortable viewing surface for the user.

According to one embodiment and when in the position for use, the user has a device with the form of a compact box with two main surfaces, wherein one surface has the receptacle with the emulsion, while the opposite surface has the mirror.

The packaging device according to the invention is typically comparable to a product of the “cream cushion” or “foundation cushion” or “compact cushion” type that are known to comprise a receptacle for a cellular sponge (called “air cushion”) soaked in a tinted cream that unifies the skin, and hydrates and protects it from ultraviolet rays. Typically, the cream may be applied using a small cushion (or “pad” or “foam disc”) that the user tamps against the powder compact.

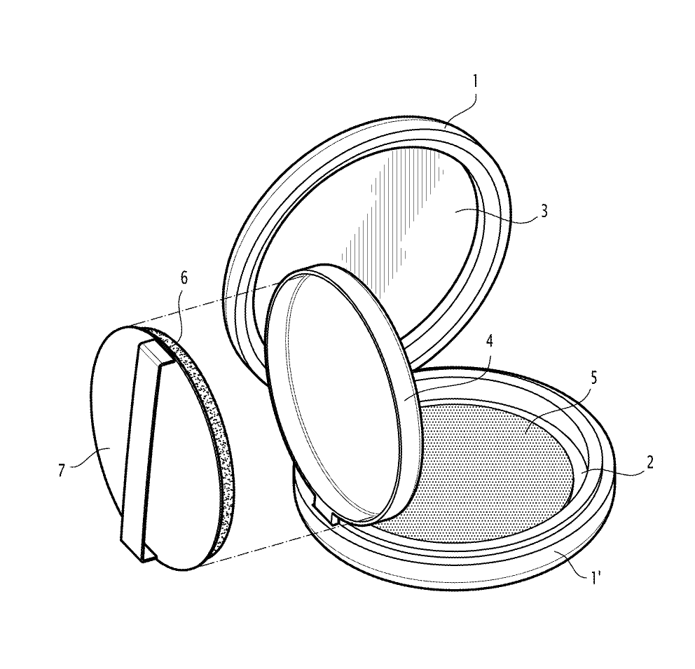

FIG. 1 depicts a preferred mode of the packaging device according to the invention, i.e. open mode for use. In particular, this round-shaped device comprises a housing (cover (1) and body (1′)), a mirror (3) located on the inner surface of the cover (1) and a receptacle comprising an element having at least one orifice (5), such as a sponge. In addition, the device according to FIG. 1 comprises a closure member (4) which is in the open position, and which may contain the applicator (6) comprising a support (7) to facilitate its being gripped.

The packaging device according to the invention may be adapted to be carried by a user, for example in a handbag. In fact, the packaging device according to the invention advantageously has a portable or “nomadic” character and is designed to be held by or in the hand. The device according to the invention is advantageous because it is small and easy to carry, and allows the user to carry it in a handbag, for example, or even in a pocket.

The applicator of the device advantageously allows easy application of the emulsion. In particular, an applicator in the form of foam advantageously offers a pleasant application, and makes it possible to regulate the dose to be taken up and applied. In addition, the controlled application of the emulsion advantageously makes it possible to play on the degrees of coverage and/or loading of the perfuming agent(s), from the most subtle to the most intense.

The presence of an applicator in the device advantageously makes it possible to avoid any contact between the user's fingers and the emulsion to be applied.

Furthermore, it has been advantageously observed that the use of a device according to the invention makes it possible to improve the sensory properties felt by the user during the application of the emulsion to a keratin material, particularly in terms of hydration, which gives a feeling of freshness and lightness (good coverage while remaining light on the skin).

In addition, the implementation of a product of the “cream cushion” type is an unprecedented alternative offered to users for the application of a perfume (or perfume composition). Moreover, a better persistence of the olfactory effect conferred by the perfuming agent(s) and an improved olfactory power could be advantageously observed during the application of an emulsion according to the invention, wherein it comprised at least one perfuming agent, by means of the packaging device according to the invention.

Emulsion

According to the invention, the emulsion comprises a continuous phase and a dispersed phase in the form of drops, wherein the drops comprise a shell formed by at least one anionic polymer and at least one cationic polymer.

According to one embodiment, the emulsion is of the water-in-oil or oil-in-water type.

According to a preferred embodiment, the emulsion is of the oil-in-water type and thus comprises a continuous aqueous phase and a dispersed fatty phase in the form of drops, wherein the drops comprise a shell formed by at least one anionic polymer and at least one cationic polymer.

In the context of the present invention, the aforementioned emulsion may be denoted by the term “dispersion”.

According to one embodiment, an emulsion according to the invention does not comprise a surfactant. It is therefore different from the usual cosmetic compositions, especially by being in emulsion form.

The emulsions according to the invention are of particular interest as regards the texture by differentiating themselves from “conventional” emulsions that are stabilized by surfactants.

In fact, the emulsions according to the invention are characterized by a unique texture that is light and voluble, providing a two-stage application. More particularly, the emulsions according to the invention spread easily on the skin. The first moments of application are very aqueous with a marked breaking effect. Then, the feeling evolves towards an oily veil that fades to leave a light and hydrated skin.

According to the invention, the pH of the emulsion is typically between 5.5 and 8.0. According to one embodiment, an emulsion according to the invention is prepared by implementing a “non-microfluidic” process, i.e. by simple emulsification. According to this embodiment, the drop size of the dispersed phase is less than 500 μm, or even less than 200 μm. Preferably, the size of the drops lies between 0.5 μm and 50 μm, preferably between 1 μm and 20 μm.

According to this embodiment, the present invention makes it possible to have drops of reduced size, especially with respect to drops obtained by a microfluidic process. This small size of drops will have an effect on the texture. In fact, an emulsion according to the invention, formed of finely dispersed drops, offers improved lubricity qualities.

In the context of the present invention, the term “size” refers to the diameter, in particular the average diameter, of the drops.

Viscosity

The viscosity of the emulsions according to the invention may vary significantly, which thus makes it possible to obtain varied textures.

According to one embodiment, the emulsion according to the invention has a viscosity of from 1 mPa·s to 500,000 mPa·s, preferably from 10 to 300,000 mPa·s, and better still 1,000 mPa·s to 100,000 mPa·s, as measured at 25° C.

The viscosity is measured at ambient temperature, for example T=25° C.±2° C. and at ambient pressure, for example 1013 mbar, by the method described below.

A Brookfield type viscometer, typically a Brookfield RVDV-E digital viscometer (torsion spring torque of 7187.0 dyne-cm) is used, which is a rotational speed viscometer provided with a movable spindle. A speed is imposed on the spindle in rotation and the measurement of the torque exerted on the spindle makes it possible to determine the viscosity by knowing the geometry/shape parameters of the spindle used.

For example, a spindle of size No. 04 (Brookfield reference: RV4) is used. The shear rate corresponding to the measurement of the viscosity is defined by the spindle used and the speed of rotation thereof.

The viscosity measurement is carried out for 1 minute at room temperature (T=25° C.±2° C.). About 150 g of solution are placed in a beaker of 250 ml volume, having a diameter of about 7 cm so that the height of the volume occupied by the 150 g of solution is sufficient to reach the gauge marked on the spindle. Then, the viscometer is started at a speed of 10 rpm and until the value displayed on the screen becomes stable. This measurement gives the viscosity of the tested fluid, as mentioned in the context of the present invention.

Fatty Phase

According to the invention, the emulsions may comprise a fatty phase, in particular dispersed, in the form of drops.

According to the invention, the fatty phase may comprise at least one H1 oil in which the cationic polymer is soluble. In fact, the emulsion according to the invention may comprise at least one oil compatible with the cationic polymer. The H1 oil therefore corresponds to a good solvent for the cationic polymer.

The term “oil” is understood to mean a fatty substance that is liquid at room temperature (25° C.).

Among H1 oils used in an emulsion according to the invention, may be mentioned, for example:
hydrocarbon oils of animal origin, such as perhydrosqualene and squalane;
esters and synthetic ethers, in particular of fatty acids, such as the oils of the formulas R₁COOR₂ and R₁OR₂ in which R₁ represents the residue of a C₈ to C₂₉ fatty acid, and R₂ represents a branched or unbranched hydrocarbon chain C₃ to C₃₀, such as, for example, purcellin oil, isononyl isononanoate, isodecyl neopentanoate, isopropyl myristate, 2-ethylhexyl palmitate, octyl-2 stearate dodecyl, octyl-2-dodecyl erucate, isostearyl isostearate; hydroxylated esters such as isostearyl lactate, octyl hydroxystearate, octyldodecyl hydroxystearate, diisostearyl malate, triisocetyl citrate, heptanoates, octanoates, decanoates of fatty alcohols; polyol esters, such as propylene glycol dioctanoate, neopentyl glycol diheptanoate and diethylene glycol diisononanoate; and pentaerythritol esters such as pentaerythrityl tetraisostearate (Prisorine 3631);
linear or branched hydrocarbons of mineral or synthetic origin, such as paraffin oils, volatile or not, and their derivatives, petroleum jelly, polydecenes, hydrogenated polyisobutene such as Parleam oil;
silicone oils, for example volatile or non-volatile polymethylsiloxanes (PDMSs) with a linear or cyclic silicone chain, which are liquid or pasty at room temperature, in particular cyclopolydimethylsiloxanes (cyclomethicones) such as cyclohexasiloxane and cyclopentasiloxane; polydimethylsiloxanes (or dimethicones) comprising alkyl, alkoxy or phenyl groups, in or at the end of the silicone chain, groups having from 2 to 24 carbon atoms; phenyl silicones such as phenyltrimethicones, phenyldimethicones, phenyltrimethylsiloxydiphenylsiloxanes, diphenyldimethicon-es, diphenylmethyldiphenyltrisiloxanes, 2-phenylethyltri-methylsiloxysilicates, and polymethylphenylsiloxanes;
fatty alcohols having from 8 to 26 carbon atoms, such as cetyl alcohol, stearyl alcohol and their mixture (cetylstearyl alcohol), or else octyldodecanol;
partially fluorinated hydrocarbon oils and/or silicone oils such as those described in document JP-A-2-295912;
and their mixtures.

According to one embodiment, the H1 oil is chosen from among the esters of formula R₁COOR₂, in which R₁ represents the residue of a C₈ to C₂₉ fatty acid, and R₂ represents a branched or unbranched hydrocarbon chain at C₃ to C₃₀.

According to one embodiment, the H1 oil is chosen from fatty alcohols having from 8 to 26 carbon atoms.

According to one embodiment, the H1 oil is chosen from among silicone oils, for example polydimethylsiloxanes (PDMS).

According to one embodiment, the H1 oil is chosen from among hydrocarbon oils having from 8 to 16 carbon atoms, and in particular C₈-C₁₆ branched alkanes (also known as isoparaffins or isoalkanes), such as isododecane (also called 2,2,4,4,6-pentamethylheptane), isodecane, isohexadecane, and, for example, the oils sold under the trade names Isopars® or Permethyls®.

According to a preferred embodiment, the H1 oil is chosen among from the group consisting of isononyl isononanoate, dimethicone, isohexadecane, polydimethylsiloxane, octyldodecanol, isodecyl neopentanoate and their mixtures.

Preferably, the H1 oil is isononyl isononanoate.

According to one embodiment, the H1 oil is not a vegetable oil.

According to one embodiment, the H1 oil is not represented by polydimethylsiloxane (PDMS), and preferably is not a silicone oil.

According to another embodiment, the fatty phase of the drops does not comprise polydimethylsiloxane (PDMS), and preferably does not comprise silicone oil.

According to a preferred embodiment, an emulsion according to the invention comprises at least 1% by weight of H1 oil(s), preferably isononyl isononanoate, relative to the total weight of the emulsion.

According to one embodiment, the content of H1 oil(s) in the fatty phase is between 1% and 99.99%, preferably between 20% and 90%, and in particular between 50% and 80%, by weight, relative to the total weight of the fatty phase.

According to one embodiment, the fatty phase of the emulsions according to the invention may further comprise at least one hydrocarbon H2 oil of plant origin. The fatty phase may comprise several H2 oils.

As H2 vegetable oils, particular mention may be made of liquid triglycerides of C₄-C₁₀ fatty acids such as triglycerides of heptanoic or octanoic acids, or else, for example, oils of sunflower, corn, soybean, squash, grapeseed, sesame, hazelnut, apricot, macadamia, arara, sunflower, castor, avocado, caprylic/capric acid triglycerides, such as those marketed by the company Stearineries Dubois or those available under the names “Miglyol 810”, “Miglyol 812” and “Miglyol 818” from the company Dynamit Nobel, jojoba oil, shea butter oil, and mixtures thereof.

Preferably, the H2 oil is chosen from among oils that are rich in polyunsaturated fatty acids.

For the purposes of the present invention, the term “unsaturated fatty acid” is understood to mean a fatty acid comprising at least one double bond. It is more particularly long-chain fatty acids, i.e. that may have more than 14 carbon atoms. The unsaturated fatty acids may be in acid form, or in salt form, such as, for example, their calcium salt, or in the form of derivatives, in particular fatty acid ester(s).

Preferably, the H2 oil is chosen from oils rich in long-chain fatty acids, i.e. that are able to have more than 14 carbon atoms, and even better unsaturated fatty acids containing from 18 to 22 carbon atoms. especially ω-3 and ω-6 fatty acids. Thus, advantageously, the vegetable oils are chosen from among evening primrose, borage, blackcurrant seed, hemp, walnut, soybean, sunflower, wheat germ, fenugreek, rosebush and musk rosebush, echium, argan, baobab, rice bran, sesame, almond, hazelnut, chia, flax, olive, avocado, safflower, coriander, rapeseed (in particular Brassica naptus), and their mixtures.

Preferably, the H2 oil is chosen from matt and non-glossy oils. In particular, mention may be made of Moringa oil.

According to one embodiment, the content of H2 oil(s) in the fatty phase lies between 0% and 40%, preferably between 0.1% and 25%, and in particular between 1% and 20%, by weight relative to the total weight of the fatty phase.

According to one embodiment, the mass ratio between the amount of H1 oil(s) and the amount of H2 oil(s) ranges from 0.025 to 99.99, preferably from 0.8 to 90, and in particular from 2.5 to 80.

The fatty phase may further comprise at least one other oil that is different from the H1 and H2 oils.

An oil-in-water emulsion according to the invention may comprise from 0.0001% to 50%, preferably from 0.1% to 40%, and better still from 1% to 25% by weight of oil(s) relative to the total weight of the emulsion.

According to one embodiment, the fatty phase of an emulsion according to the invention may comprise at least one fatty substance that is solid at ambient temperature and pressure, in particular chosen from among waxes, pasty fatty substances, butters, and mixtures thereof.

Wax(es)

For the purposes of the invention, the term “wax” is understood to mean a lipophilic compound, solid at room temperature (25° C.), with a reversible solid/liquid state change, having a melting point greater than or equal to 30° C. up to 120° C. The melting point may be measured according to the protocol described in FR 15 58849.

The waxes that may be used in an emulsion according to the invention are chosen from among solid waxes that are deformable or not at room temperature, and are of animal, vegetable, mineral or synthetic origin, and mixtures thereof.

In particular, it is possible to use hydrocarbon-based waxes such as beeswax, lanolin wax, and Chinese insect waxes; rice wax, Carnauba wax, Candelilla wax, Ouricurry wax, Alfa wax, cork fiber wax, sugar cane wax, Japanese wax and sumac wax; montan wax, microcrystalline waxes, paraffins and ozokerite; polyethylene waxes, waxes obtained by Fisher-Tropsch synthesis as well as their waxy copolymers and their esters, and mixtures thereof.

Mention may also be made of waxes obtained by catalytic hydrogenation of animal or vegetable oils having linear or branched C₈-C₃₂ fatty chains.

Among these, may be mentioned hydrogenated jojoba oil, hydrogenated sunflower oil, hydrogenated castor oil, hydrogenated coconut oil and hydrogenated lanolin oil, di-tetrastearate(trimethylol-1,1,1 propane) sold under the name “HEST 2T-4S” by the company HETERENE, di-(1,1,1-trimethylolpropane)tetraprenate sold under the name HEST 2T-4B by the company HETERENE.

It is also possible to use the waxes obtained by transesterification and hydrogenation of vegetable oils, such as castor oil or olive oil, and such as the waxes sold under the names Phytowax ricin 16L64® and 22L73® and Phytowax Olive 18L57 by the company Sophim. Such waxes are described in the application FR-A-2792190.

It is also possible to use silicone waxes, which may advantageously be substituted by polysiloxanes, preferably at a low melting point.

Among the commercial silicone waxes of this type, mention may be made, in particular, of those sold under the names Abilwax 9800, 9801 or 9810 (GOLDSCHMIDT), KF910 and KF7002 (SHIN ETSU), or 176-1118-3 and 176-11481 (GENERAL ELECTRIC).

The silicone waxes that may be used may also be alkyl or alkoxydimethicones such as the following commercial products: Abilwax 2428, 2434 and 2440 (GOLDSCHMIDT), or VP 1622 and VP 1621 (WACKER), as well as (C₂₀-C₆₀) alkyldimethicones, in particular (C₃₀-C₄₅) alkyldimethicones such as the silicone wax sold under the name SF-1642 by the company GE-Bayer Silicones.

It is also possible to use hydrocarbon waxes modified with silicone or fluorinated groups such as, for example: siliconyl candelilla, siliconyl beeswax and Fluorobeeswax by Koster Keunen.

The waxes may also be chosen from fluorinated waxes.

Butter or Pasty Fat

For the purposes of the present invention, the term “butter” (also referred to as “pasty fatty substance”) is understood to mean a lipophilic fatty compound with a reversible solid/liquid state change and comprising a liquid fraction and a solid fraction at the temperature of 25° C. and atmospheric pressure (760 mmHg).

The pasty fatty substance or butter may be chosen from among synthetic compounds and compounds of plant origin. A pasty fatty substance may be obtained synthetically from starting materials of plant origin.

The pasty fatty substance is advantageously chosen from among:
lanolin and its derivatives such as lanolin alcohol, oxyethylenated lanolines, acetylated lanolin, lanolin esters such as isopropyl lanolate, oxypropylenated lanolines,
the polymeric or non-polymeric silicone compounds, such as polydimethylsiloxanes of high molecular weight, poly-dimethylsiloxanes with side chains of the alkyl or alkoxy type having from 8 to 24 carbon atoms, especially stearyl dimethicones,
polymeric or non-polymeric fluorinated compounds,
vinyl polymers, in particular
homopolymers of olefins,
the olefin copolymers, homopolymers and copolymers of hydrogenated dienes,
linear or branched oligomers, homo or copolymers of alkyl (meth) acrylates preferably having a C₈-C₃₀ alkyl group,
homo and copolymeric oligomers of vinyl esters having C₈-C₃₀ alkyl groups,
homo and copolymer oligomers of vinyl ethers having C₈-C₃₀ alkyl groups,
the liposoluble polyethers resulting from polyetherification between one or more C₂-C₁₀₀ and preferably C₂-C₅₀ diols,
esters and polyesters, and
their mixtures.

As vegetable butters, mention may be made of those described in Ullmann's Encyclopedia of Industrial Chemistry (“Fats and Fatty Oils”, A. Thomas, published on 15 Jun. 2000, D01: 10.1002/14356007.a10_173, point 13.2.2.2). Shea Butter, Borneo Tallow, and Related Fats (Vegetable Butters). One may mention more particularly triglycerides C₁₀-C₁₈ (INCI name: C10-18 Triglycerides) comprising a liquid fraction and a solid fraction at a temperature of 25° C. and at atmospheric pressure (760 mm Hg), shea butter, Nilotica Shea butter (Butyrospermum parkii), Galam butter, (Butyrospermum parkii), Borneo butter or fat or Tengkawang tallow) (Shorea stenoptera), Shorea butter, Illipé butter, Madhuca butter or Bassia Madhuca longifolia, mowrah butter (Madhuca Latifolia), Katiau butter (Madhuca mottleyana), Phulwara butter (M. butyracea), mango butter (Mangifera indica), Murumuru butter (Astrocatyum murumuru), Kokum butter (Garcinia Indica), Ucuuba butter (Virola sebifera), Tucuma butter, Painya butter (Kpangnan) (Pentadesma butyracea), Coffee butter (Coffea arabica), Apricot butter (Prunus Armeniaca), Macadamia butter (Macadamia Temifolia), butter grape (Vitis vinifera), avocado butter (Persea gratissima), olive butter (Olea europaea), sweet almond butter (Prunus amygdalus dulcis), cocoa butter (Theobroma cacao) and sunflower butter, butter under the INCI name Astrocaryum Murumuru Seed Butter, butter under the INCI name Theobroma Grandiflorum Seed Butter, and butter under the INCI name Irvingia Gabonensis Kernel Butter, jojoba esters (mixture of wax and oil hydrogenated jojoba) (INCI name: Jojoba esters) and ethyl esters of shea butter (INCI name: Shea butter ethyl esters), and mixtures thereof.

Preferably, an emulsion according to the invention may comprise from 0% to 98.99% by weight, preferably from 0.5% to 70% by weight, in particular from 1% to 30% by weight, and better still from 1% to 20% by weight, of solid fatty substance(s) relative to the total weight of the fatty phase.

According to one embodiment where the emulsion according to the invention comprises at least one perfuming agent, the fatty phase may be devoid of oil and/or solid fatty substance. In fact, certain perfuming agents, besides their perfuming action, also act as a solvent for the cationic polymer, in particular amodimethicone.

Drops Shell

As mentioned above, the drops according to the invention are surrounded by a shell (also referred to as a “membrane”).

According to the invention, the drops so obtained may have a very thin shell, in particular a thickness less than 1% of the diameter of the drops.

The thickness of the shell is thus preferably less than 1 μm and is too thin to be measured by optical methods.

According to one embodiment, the thickness of the shell of the drops is less than 1000 nm, in particular between 1 and 500 nm, preferably less than 100 nm, advantageously less than 50 nm, and preferably less than 10 nm.

The measurement of the thickness of the shell of the drops of the invention may be carried out by the small angle X-ray scattering method, as implemented in Sato et al. J. Chem. Phys. 111, 1393-1401 (2007).

For this purpose, the drops are produced using deuterated water and are then washed three times with a deuterated oil, such as, for example, a deuterated hydrocarbon-type oil (octane, dodecane, hexadecane).

After washing, the drops are then transferred to the neutrons cell to determine the I(q) spectrum, wherein q is the wave vector.

From this spectrum, conventional analytical treatments (REF) are applied to determine the thickness of the hydrogenated (undeuterated) shell.

According to one embodiment, the shell surrounding the drops of the dispersed phase is stiffened, which, in particular, gives good resilience to the drops, and reduces, or even prevents, their coalescence.

This shell is typically formed by coacervation, i.e. precipitation of charged polymers of opposite charges. Within a coacervate, the bonds binding the charged polymers to each other are of ionic type, and are generally stronger than bonds present within a surfactant-type membrane.

The shell is formed by coacervation of at least two charged polymers of opposite polarity (or polyelectrolyte) and preferably in the presence of a first polymer of the cationic type, and a second polymer, different from the first polymer, of the anionic type. These two polymers act as stiffening agents for the membrane.

The formation of the coacervate between these two polymers is generally caused by a modification of the conditions of the reaction medium (temperature, pH, reagent concentration, etc.). The reaction of the coacervation results from the neutralization of these two charged polymers of opposite polarities, and allows the formation of a membrane structure by electrostatic interactions between the anionic polymer and the cationic polymer. The membrane thus formed around each drop typically forms a shell which completely encapsulates the heart of the drop and thus isolates the core of the drop from the continuous aqueous phase.

Anionic Polymer

In the context of the present invention, the term “anionic polymer” (or “anionic-type polymer”) is understood to mean a polymer having the chemical functions of anionic type. We may also speak of anionic polyelectrolyte.

By “anionic chemical function” is meant an AH chemical function capable of giving a proton to give a function A. Depending on the conditions of the medium in which it is found, the anionic type polymer therefore has chemical functions in AH form, or in the form of its conjugate base A.

As an example of chemical functions of the anionic type, mention may be made of the carboxylic acid functions —COOH, optionally present in the form of carboxylate anion —COO—.

As an example of anionic type polymer, we may mention any polymer formed by the polymerization of monomers, at least a part of which carries anionic type chemical functions, such as carboxylic acid functions. Such monomers are, for example, acrylic acid, maleic acid, or any ethylenically unsaturated monomer containing at least one carboxylic acid function. It may be, for example, an anionic polymer comprising monomeric units comprising at least one chemical function of the carboxylic acid type.

Preferably, the anionic polymer is hydrophilic, i.e., soluble or dispersible in water.

According to one embodiment, the continuous phase, preferably the continuous aqueous phase, comprises at least one anionic polymer.

Examples of anionic polymer suitable for carrying out the invention include copolymers of acrylic acid or maleic acid and other monomers, such as acrylamide, alkyl acrylates, C₅-C₈ alkyl acrylates, C₁₀-C₃₀ alkyl acrylates, C₁₂-C₂₂ alkyl methacrylates, methoxypoly-ethylene glycol methacrylates, hydroxyester acrylates, crosspolymer acrylates, and mixtures thereof.

According to one embodiment, the anionic polymer according to the invention is a crosslinked carbomer or copolymer acrylates/C₁₀₋₃₀ alkyl acrylate. Preferably, the anionic polymer according to the invention is a carbomer.

According to one embodiment, the shell of the drops comprises at least one anionic polymer, such as for example a carbomer.

In the context of the invention, and unless otherwise stated, the term “carbomer” refers to an optionally crosslinked homopolymer resulting from the polymerization of acrylic acid. It is therefore a poly (acrylic acid) that is optionally crosslinked.

Among the carbomers of the invention, mention may be made of those sold under the names Tego® Carbomer 340FD from Evonik, Carbopol® 981 from Lubrizol, Carbopol ETD 2050 from Lubrizol or Carbopol Ultrez 10 from Lubrizol.

According to one embodiment, the term “carbomer” or Carbopol® refers to a high molecular weight acrylic acid polymer crosslinked with allyl sucrose or pentaerythritol allyl ethers (handbook of Pharmaceutical Excipients, 5th Edition, pill). Examples include Carbopol® 910, Carbopol® 934, Carbopol® 934P, Carbopol® 940, Carbopol® 941, Carbopol® 71G, Carbopol® 980, Carbopol® 971P or Carbopol® 974P. According to one embodiment, the viscosity of the carbomer lies between 4,000 and 60,000 cP at 0.5% w/w.

The carbomers also have other names: polyacrylic acids, carboxyvinyl polymers or carboxy polyethylenes.

According to the invention, the aforementioned dispersion may comprise from 0.01% to 5%, preferably from 0.05% to 2%, and preferably from 0.10% to 0.5%, by weight of anionic polymer(s), especially carbomer(s), relative to the total weight of the dispersion.

Cationic Polymer

The drops, and in particular the shell of the drops, comprise a cationic type polymer. They may also comprise several cationic type polymers. This cationic polymer is the one mentioned above and which forms the shell by coacervation with the anionic polymer.

In the context of the present application, and unless otherwise stated, the term “cationic polymer” (or “cationic type polymer”) refers to a polymer having chemical functions of a cationic type. We may also speak of cationic polyelectrolyte.

Preferably, the cationic polymer is lipophilic or fat-soluble.

According to one embodiment, the fatty phase of an emulsion according to the invention, in particular the dispersed fatty phase, comprises at least one cationic polymer.

In the context of the present application, and unless otherwise stated, “chemical function of the cationic type” is understood to mean a chemical function B capable of capturing a proton to give a function BH⁺. Depending on the conditions of the medium in which it is located, the cationic type polymer therefore has chemical functions in the form B or in the form BH⁺, its conjugated acid.

As an example of chemical functions of the cationic type, mention may be made of the primary, secondary and tertiary amine functions, optionally present in the form of ammonium cations.

An example of a cationic polymer that may be mentioned is any polymer formed by the polymerization of monomers, at least a part of which carries chemical functions of the cationic type, such as primary, secondary or tertiary amine functions.

Such monomers are, for example, aziridine, or any ethylenically unsaturated monomer containing at least one primary, secondary or tertiary amine function.

Examples of cationic polymers suitable for the implementation of the invention include amodimethicone, derived from a silicone polymer (polydimethylsiloxane, also called dimethicone), modified by primary amine functions and secondary amine.

Mention may also be made of amodimethicone derivatives, for example copolymers of amodimethicone, aminopropyl dimethicone, and, more generally, linear or branched silicone polymers containing amine functional groups.

The bis-isobutyl PEG-14/amodimethicone copolymer, the bis (C13-C15 Alkoxy) PG-Amodimethicone, the bis-Cetearyl amodi-methicone and bis-hydroxy/methoxy amodimethicone may be mentioned.

Mention may also be made of polysaccharide polymers comprising amine functions, such as chitosan or guar gum derivatives (hydroxypropyltrimonium guar chloride).

Mention may also be made of polypeptide polymers comprising amine functions, such as polylysine.

Mention may also be made of polyethyleneimine polymers comprising amine functions, such as linear or branched poly-ethyleneimine.

According to one embodiment, the drops, and, in particular, the shell of the drops, comprise(s) a cationic polymer which is a silicone polymer modified with a primary, secondary or tertiary amine function, such as amodimethicone.

According to one embodiment, the drops, and, in particular, the shell of the drops, comprise amodimethicone.

According to a particularly preferred embodiment, the cationic polymer has the following formula:

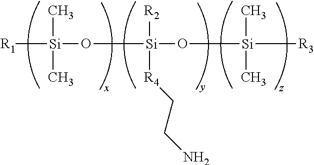

wherein:
R₁, R₂ and R₃, independently of each other, represent OH or CH₃;
R₄ represents a group —CH₂— or a group —X—NH— in which X is a divalent alkylene radical C3 or C4;
x is an integer between 10 and 5000, preferably between 30 and 1000, and more preferably between 80 and 300;
y is an integer between 2 and 1000, preferably between 4 and 100, and more preferably between 5 and 20; and
z is an integer between 0 and 10, preferably between 0 and 1, and better is equal to 1.

In the aforementioned formula, when R₄ is —X—NH—, X is attached to the silicon atom.

In the aforementioned formula, R₁, R₂ and R₃ preferably represent CH₃.

In the aforementioned formula, R₄ is preferably a —(CH₂)₃—NH— group.

According to the invention, each drop may comprise from 0.01% to 10%, preferably from 0.05% to 5%, by weight of cationic polymer(s), in particular of amodimethicone(s), relative to the total weight of the fatty phase.

Aqueous Phase

In addition to the anionic polymer as defined above, the aqueous phase, especially continuous emulsions according to the invention, may comprise water.

Preferably, when the aqueous phase of an emulsion according to the invention is the continuous phase, the latter is advantageously in the form of a gel.

In addition to distilled or deionized water, water suitable for the invention may also be natural spring water or floral water.

According to one embodiment, the mass percentage of water of the aqueous continuous phase is at least 40%, and better still at least 50%, especially between 70% and 98%, preferably between 75% and 95%, relative to the total mass of the continuous phase.

An emulsion according to the invention may comprise at least 20%, preferably at least 30%, in particular at least 40%, and better still at least 50% by weight, of water relative to the total weight of the emulsion.

Preferably, the emulsions according to the invention comprise at least 75% by weight of aqueous phase.

Buffer

The aqueous phase, especially the continuous aqueous phase, of an emulsion according to the invention may further comprise at least one buffer. According to the invention, the buffer used has a pKa of from 4.0 to 9.0.

In the context of the present invention, and unless otherwise indicated, the term “buffer” is understood to mean a chemical species which, in aqueous solution, maintains the pH of the aqueous composition in which it is solubilized, despite the addition of small amounts acid or base, or despite dilution.

According to one embodiment, the buffer comprises one or two sulfonic acid functions, preferably only one.

Preferably, the pKa of the buffer is between 5.0 and 8.0, preferably between 6.0 and 8.0.

According to one embodiment, the buffer is selected from the group consisting of phosphate buffers, 2-(N-morpholino)ethanesulfonic acid (MES), 2-amino-2-hydroxymethyl-1,3-propanediol, 2-(bis(2-5-hydroxyethyl)amino)acetic acid, 4-(2-hydroxyethyl)-1-piperazine ethane sulfonic acid (HEPES), sodium citrate and mixtures thereof.

In the context of the present invention, and unless otherwise indicated, “phosphate buffer” is understood to mean a buffer comprising dihydrogenphosphate and hydrogenphosphate ions.

A phosphate buffer according to the invention may be prepared by dissolving monosodium or monopotassium phosphate and disodium or dipotassium phosphate in water.

As phosphate buffer, mention may be made of PBS, which denotes phosphate buffered saline that is prepared by dissolving disodium phosphate (10 mM), monopotassium phosphate (1.76 mM), chloride sodium (137 mM) and potassium chloride (2.7 mM) in water. PBS has a pKa of 7.2 and is used to buffer an aqueous composition in a pH range of 6.5 to 7.9.

As a phosphate buffer, mention may also be made of the buffer prepared by dissolving disodium phosphate (0.44% by weight) and monopotassium phosphate (2.74% by weight) in water. Such a buffer has a pKa of 5.8.

In particular, the buffer is 4-(2-hydroxyethyl)-1-piperazine ethane sulfonic acid, in particular called HEPES (CAS No. 7365-45-9). HEPES has a pKa of 7.5 and is used to buffer an aqueous composition in a pH range of 6.8 to 8.2.

According to one embodiment, the aqueous phase, in particular the continuous aqueous phase, of an emulsion according to the invention may comprise from 0.1% to 10%, preferably from 0.5% to 5% by weight of buffer(s) relative to the total weight of the aqueous phase.

The emulsion according to the invention, in particular of the oil-in-water type, may comprise from 0.1% to 10% by weight of buffer(s), preferably from 0.5% to 5% by weight, relative to the total weight of the emulsion.

Base

The continuous aqueous phase of the emulsion according to the invention may further comprise at least one base. It may comprise a single base or a mixture of several different bases. The presence of at least one base in the aqueous continuous phase contributes, in particular, to enhancing the viscosity of the latter.

According to one embodiment, the base present in the aqueous continuous phase is a mineral base, preferably selected from the group consisting of alkali metal hydroxides and alkaline earth metal hydroxides.

Preferably, the mineral base is an alkali metal hydroxide, and especially NaOH.

According to one embodiment, the base present in the aqueous continuous phase is an organic base. Among the organic bases, mention may be made, for example, of ammonia, pyridine, triethanolamine, aminomethylpropanol, or else triethylamine.

An oil-in-water type emulsion according to the invention may comprise from 0.01% to 10% by weight, preferably from 0.01% to 5% by weight, and more preferably from 0.02% to 1% by weight of base, preferably of mineral base, and in particular of NaOH, relative to the total weight of the emulsion.

Perfuming Agent

According to one embodiment, the emulsions according to the invention may further comprise at least one perfuming agent.

According to this embodiment, and as is apparent from the examples below, the application by means of a packaging device according to the invention of an emulsion according to the invention comprising at least one perfuming agent, in addition to the aforementioned advantages, offers:
a better persistence of the olfactory effect conferred by the perfuming agent present in the emulsion, and
improved olfactory power.

Without wishing to be bound to any theory, the improved olfactory performances observed with the device according to the invention may result in the absence of denaturation of the perfume that may possibly occur in the case of “conventional” modes of application, i.e. especially of the spray type, or with an application in contact with the fingers of the user. The emulsion according to the invention may comprise at least 2% by weight, in particular at least 3% by weight, preferably from 3% to 35% by weight, in particular from 5% to 20% by weight, and better still 5% to 15% by weight, and more preferably 5% to 10% by weight, of perfuming agent(s) relative to the total weight of the emulsion.

According to one embodiment, the content of perfuming agent(s) is at least 3%, preferably at least 5% by weight relative to the total weight of the emulsion. In particular, the emulsion comprises from 5% to 10% by weight of perfuming agent(s) relative to the total weight of the emulsion.

According to the invention, the perfuming agent or perfume may be in the form of a mixture. Thus, the drops according to the invention may comprise a single perfuming agent (or a single perfume) or a mixture of several perfuming agents (or a mixture of several perfumes).

Among the perfuming agents, mention may be made of any type of perfume or fragrance, wherein these terms are used here indifferently. These perfumes or fragrances are well known to persons skilled in the art and include, in particular, those mentioned, for example, in S. Arctander, Perfume and Flavor Chemicals (Montclair, N J, 1969), S. Arctander, Perfume and Flavor Materials of Natural Origin (Elizabeth, N.J., 1960) and in “Flavor and Fragrance Materials” 1991 (Allured Publishing Co. Wheaton, Ill. USA). The perfumes used in the context of the present invention may include natural products such as extracts, essential oils, absolutes, resinoids, resins, concretes, etc. as well as basic synthetic substances such as hydrocarbons, alcohols aldehydes, ketones, ethers, acids, esters, acetals, ketals, nitriles, etc., including saturated and unsaturated compounds, aliphatic, alicyclic and heterocyclic compounds.

According to one embodiment, the perfuming agent comprises less than 25% by weight of alcohol(s), linear or branched, saturated or optionally comprising at least one unsaturation, relative to the total weight of the perfuming agent. More particularly, according to one embodiment, the perfuming agent may comprise less than 25% by weight of terpenic alcohol(s) relative to the total weight of the perfuming agent.

According to one embodiment, the perfuming agent comprises less than 10%, or even less than 5%, by weight of aldehyde(s), relative to the total weight of the perfuming agent.

According to one embodiment, the perfuming agent comprises less than 10%, or even less than 7.5%, by weight of compound(s) with a ClogP less than 2.1, relative to the total weight of the perfuming agent.

According to one embodiment, the perfuming agent comprises at least 3%, or even at least 4%, by weight of alcohol(s), linear and/or branched, and less than 25%, or even less than 20%, or even less than 15%, by weight of alcohol(s), linear and/or branched, relative to the total weight of the perfuming agent.

According to one embodiment, the perfuming agent comprises 4% by weight of alcohol(s), linear and/or branched, relative to the total weight of the perfuming agent.

According to one embodiment, the perfuming agent comprises 13% by weight of alcohol(s), linear and/or branched, relative to the total weight of the perfuming agent.

According to one embodiment, the perfuming agent does not comprise aldehyde.

According to one embodiment, the perfuming agent does not comprise a compound with a Clog P of less than 2.1.

According to one embodiment, a drop of an emulsion comprising at least one perfuming agent according to the invention comprises more than 60%, even more than 70%, preferably more than 80%, and more preferably more than 90%, by weight of perfuming agent(s) relative to the total weight of the drop.

Preferably, the perfuming agent(s) is/are present in the fatty phase or in the dispersed phase of an emulsion according to the invention, and more preferably in the dispersed fatty phase.

Thus, according to the invention, the dispersed phase, in particular the dispersed fatty phase, of an emulsion according to the invention comprises at least 3% by weight, preferably at least 5% by weight of perfuming agent(s) relative to the total weight of the dispersed phase.

Crosslinked Polymer/Copolymer

According to one embodiment, an emulsion according to the invention, preferably of the oil-in-water type, and in particular the aqueous phase of the emulsion, may further comprise at least one crosslinked polymer or at least one crosslinked copolymer, wherein the crosslinked polymer or crosslinked copolymer comprises at least one unit derived from the polymerization of one of the following monomers: acrylic or methacrylic acid, alkyl acrylate or methacrylate comprising from 1 to 30 carbon atoms, or their salts (hereinafter referred to as cross-linked polymer/copolymer), that are different from the anionic and cationic polymers described above.

The presence of such a crosslinked polymer/copolymer is particularly advantageous in the case of an emulsion according to the invention comprising at least one perfuming agent, especially when present in the dispersed phase. In fact, the presence of such a crosslinked polymer/copolymer contributes to the stabilization of the perfuming agent in the emulsion and especially over a period of time greater than one month, and at temperatures of between 5° C. and 50° C., preferably between 10° C. and 60° C.

According to the invention, the term “unit derived from the polymerization of a monomer” is understood to mean that the polymer or copolymer is a polymer or copolymer that is obtained by polymerization or copolymer of the monomer.

According to one embodiment, the crosslinked polymer or the crosslinked copolymer is a crosslinked polyacrylate.

The crosslinked copolymers and polymers of the invention are anionic.

According to one embodiment, the copolymer is an unsaturated carboxylic acid copolymer and unsaturated C₁₋₃₀, preferably C₁-C₄, alkyl carboxylate. Such a copolymer comprises at least one hydrophilic unit of the olefinic unsaturated carboxylic acid type and at least one hydrophobic unit of the (C₁-C₃₀) alkyl ester type of unsaturated carboxylic acid.

Preferably, these copolymers are chosen from among those whose hydrophilic unit of unsaturated olefinic carboxylic acid type corresponds to the monomer of the following formula (I):

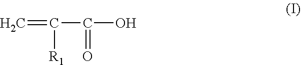

wherein: R₁ denotes H or CH₃ or C₂H₅, i.e. units of acrylic acid, methacrylic acid or ethacrylic acid, and in which the hydrophobic unit of the (C₁-C₃₀) alkyl ester of unsaturated carboxylic acid type corresponds to the monomer of the following formula (II):

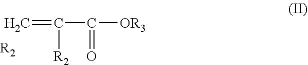

wherein: denotes H or CH₃ or C₂H₅, (i.e. acrylate, methacrylate or ethacrylate units) and preferably H (acrylate units) or CH₃ (methacrylate units), R₃ denotes a C₁-C₃₀alkyl radical; and preferably C₁-C₄.

Among this type of copolymer, those formed from a monomer mixture comprise:
(i) essentially acrylic acid,
(ii) an ester of formula (II) described above and in which R₂ denotes H or CH₃, while R₃ denotes an alkyl radical having from 1 to 4 carbon atoms, and
(iii) a crosslinking agent, which is a well-known copolymerizable polyethylenic unsaturated monomer, such as diallyl phthalate, trimethylolpropane tri(meth) acrylate, diallyl itaconate, diallyl fumarate, diallyl maleate, zinc (meth)acrylate, allyl (meth)acrylate, divinylbenzene, (poly)ethyleneglycol dimethacrylate, methylenebisacrylamide, and castor oil.

According to one embodiment, the polymer or copolymer is a polymer or copolymer of acrylic acid and/or methacrylic acid and/or alkyl acrylate comprising from 1 to 30 carbon atoms, preferably from 1 to 4 carbon atoms, and/or alkyl methacrylate comprising 1 to 30 carbon atoms, preferably 1 to 4 carbon atoms.

According to one embodiment, the crosslinked copolymer is a crosslinked copolymer of methacrylic acid and of alkyl acrylate comprising from 1 to 4 carbon atoms, preferably 2 carbon atoms.

In the context of the invention, and unless otherwise stated, the term “crosslinked copolymer of methacrylic acid and of alkyl acrylate comprising from 1 to 4 carbon atoms”, refers to a crosslinked copolymer resulting from the polymerization of a monomer of methacrylic acid and an alkyl acrylate monomer comprising from 1 to 4 carbon atoms.

Preferably, in this copolymer, the methacrylic acid is from 20% to 80% by weight, preferably from 35% to 65% by weight relative to the total weight of the copolymer.

Preferably, in this copolymer, the alkyl acrylate is from 15% to 80% by weight, preferably from 35% to 65% by weight relative to the total weight of the copolymer. In particular, the alkyl acrylate is chosen from alkyl methacrylate, ethyl acrylate and butyl acrylate.

According to one embodiment, the crosslinked polymer or the crosslinked copolymer according to the invention, present in the continuous aqueous phase, is chosen from the group consisting of the following polymers or copolymers: Acrylates Copolymer, Acrylates crosspolymer-4, Acrylates crosspolymer-3, Polyacrylate-2 Crosspolymer and Polyacrylate-14 (INCI names).

Among the above polymers, the products sold by LUBRIZOL under the trade names Fixate Superhold (INCI name=Polyacrylate-2 Crosspolymer), Fixate Freestyle Polymer (INCI name=Acrylates crosspolymer-3) are particularly preferred according to the present invention.), Carbopol® Aqua SF1 (INCI name=Acrylates copolymer) and Carbopol® Aqua SF2 (INCI name=Acrylates crosspolymer-4).

Preferably, the crosslinked polymer or the crosslinked copolymer is chosen from Carbopol® Aqua SF1 (INCI name=Acrylates copolymer) and/or Carbopol® Aqua SF2 (INCI name=Acrylates crosspolymer-4).

In particular, it is Carbopol® Aqua SF1 (INCI name=Acrylates copolymer).

According to one embodiment, the crosslinked copolymer is chosen from crosslinked copolymers of acrylic or methacrylic acid and of alkyl acrylates comprising from 1 to 4 carbon atoms.

According to the invention, the dispersion of the invention may comprise from 0.1% to 10% by weight, preferably from 0.5% to 8% by weight, and preferably from 1% to 3% by weight of crosslinked polymer(s) or crosslinked copolymer(s) relative to the total weight of the dispersion.

Texturizing Agent(s)

An emulsion according to the invention may further comprise one or more texturizing agents that are different from the cationic polymers, anionic polymers and, where present, the crosslinked polymer/copolymer described above. For the purposes of the invention, the term “texturizing agent” is understood to mean any compound intended to modulate the consistency and the touch, to maintain or modify the texture of an emulsion according to the invention.

Of course, persons skilled in the art will take care to choose the texturizing agent(s) and/or their quantity as a function of the aqueous or oily nature of the phase in question and/or in such a way that (i) the advantageous properties of an emulsion according to the invention, and (ii) the integrity of the drops forming the emulsion are not, or not substantially, impaired by the addition envisaged. These adjustments are within the competence of the person skilled in the art.

Thus, in an emulsion according to the invention, the aqueous phase may comprise at least one texturizing agent and/or the fatty phase may comprise a texturizing agent that is different from the anionic polymer, the cationic polymer and, where present, the crosslinked polymer/copolymer.

As hydrophilic texturizing agents, i.e. those which are soluble or dispersible in water, and therefore may be present in the aqueous phase of a composition according to the invention, mention may be made of:
natural texturizing agents, chosen in particular from algae extracts, plant exudates, seed extracts, exudates of microorganisms, and other natural agents,
semi-synthetic texturizing agents, especially chosen from cellulose derivatives and modified starches,
synthetic texturizing agents, especially chosen from homopolymers of (meth) acrylic acid or one of their esters, (meth) acrylic acid copolymers or one of their esters, copolymers of AMPS (2-acrylamido); 2-methylpropanesulfonic acid), associative polymers,
the other texturizing agents, especially chosen from polyethylene glycols (sold under the name Carbowax), clays, silicas such as those sold under the names Aerosil® 90/130/150/200/300/380), glycerin, and
their mixtures.

For the purposes of the present invention, the term “associative polymer” is understood to mean any amphiphilic polymer comprising in its structure at least one fatty chain and at least one hydrophilic portion, wherein the associative polymers according to the present invention may be anionic, cationic, nonionic or amphoteric, wherein they are in particular those described in FR 2 999 921. Preferably, these are amphiphilic and anionic associative polymers and amphiphilic and nonionic associative polymers as described below.

Among the natural texturizing agents, mention may be made more particularly of algae extracts represented by agar-agar, carrageenans, alginates, and mixtures thereof.

Among the natural texturizing agents, mention may be made more particularly of the exudates of plants represented by gum tragacanth, Karaya gum, gatty gum, arabic gum, and mixtures thereof.

Among the natural texturizing agents, mention may be made more particularly of the seed extracts represented by locust bean gum, guar gum, tara gum, konjac gum, pectins, and mixtures thereof.

Among the natural texturizing agents, mention may be made more particularly of the exudates of microorganisms represented by xanthan gum, gellan gum, pullulan, and mixtures thereof.

Among the natural texturizing agents, mention may also be made of other natural agents represented in particular by gelatin, collagen, keratin, plant proteins, in particular wheat and/or soy, chitin or anionic chitosan polymers, cationic, nonionic or amphoteric, hyaluronic acid or a salt thereof, especially sodium hyaluronate such as that sold under the names HA Oligo, SC Hyaluronic Acid or HyaCare, and mixtures thereof.

Among the semi-synthetic texturizing agents, the cellulose derivatives are in particular represented by carboxymethylcellulose (CMC) such as that sold under the names Aqualon series or Walocel series; hydroxypropylcellulose (HPC) such as that sold under the name Klucel HPC; hydroxyethylcellulose (HEC) such as that sold under the names Cellosize series or Natrosol 250 series; hydroxyethyl methylcellulose such as that sold under the name Walocel series; hydroxypropyl methylcellulose such as that sold under the names Methocel E/F/J/K series from Dow Chemicals, VIVAPHARM CS 152 HV, Benecel E4M, E10M, K100M; methylcellulose such as that sold under the name Methocel A series; ethylcellulose such as that sold under the name Ethocel series; microcrystalline cellulose such as that sold under the name Avicel PH series; alkylhydroxyethylcellulose such as cetylhydroxyethylcellulose sold under the name Polysurf 67), and mixtures thereof.

Among the semi-synthetic texturizing agents, the modified starches are starch derivatives resulting from the modification of the native starch by etherification, esterification or crosslinking, such as in particular sodium carboxymethyl starch such as that marketed under the names COVAGEL, VIVASTAR® CS 352 SV or VIVASTAR CS 302 SV; hydroxypropyl starch such as that sold under the names Zeina B860, Amaze NI, Amycol SQ, Penon PKW; hydroxypropyl starch phosphate such as that sold under the names Structure ZEA/style/XL; and their mixtures.

Among the synthetic texturizing agents, the homopolymers of (meth) acrylic acid or an ester thereof are, in particular, represented by sodium polyacrylates such as those sold under the names Cosmedia SP, Covacryl MV60/MV40, Cosmedia SPL or Luvigel EM; cross-linked (meth) acrylic acid polymers (or carbomers) that are different from the carbomers defined as the above anionic polymer, such as those sold under the names Carbopol 900 series, Carbopol 2984/5984, Carbopol Ultrez 10/30, in particular Carbopole Ultrez 21, Tego Carbomer 134/140/141, Aqupec HV-505, HV-505HC, HV-504, HV-501, HV-505E, HV-504E, HV-501E, HV-505ED, Ashland 941 carbomer, or Ashland 981 carbomer; and their mixtures.

Among the synthetic texturizing agents, the copolymers of (meth) acrylic acid or one of their esters are especially represented by glyceryl acrylate/acrylic acid copolymer such as that sold under the names Lubrajel series, Lubrasil series or Norgel; acrylate copolymers such as those sold under the names or Carbopol Aqua SF-1 OS Polymer (INCI name=Acrylates copolymer); crosspolymer-2 sodium acrylates such as that sold under the name Aquakeep 10 SH NF; acrylates/C₁₀-C₃₀ alkyl acrylate crosspolymers such as those sold under the names Carbopol 1342/1382, Carbopol ETD 2020, Pemulen TR-1/TR-2, Carbopol Ultrez 20/21, Tego Carbomer 341 ER, Tego Carbomer 750 HD, Tego Carbomer 841 SER, Aqupec HV-501ER, HV-701EDR, HV-501EM, SER W-150C or SER W-300C; sodium acrylates/beheneth-25 methacrylate crosspolymer such as that sold under the name Novemer EC-2; acrylates/acrylamide copolymers such as that sold under the name Novemer EC-1 by Lubrizol; acrylamide/sodium acrylate copolymers such as that sold under the name Aquagel 55; Acrylic Acid/VP crosspolymers such as that sold under the name Ultrathix P-100; and their mixtures.

Among the synthetic texturizing agents, the AMPS copolymers are in particular represented by AMPS NH 4/vinylpyrrolidone copolymers, such as that sold under the name Aristoflex AVC (INCI: Ammonium Acryloyldimethyltaurate/VP Copolymer); the NH 4/Beheneth-25 methacrylate AMPS copolymer, such as the product sold under the name Aristoflex HMB (INCI: Ammonium Acryloyl-dimethyltaurate/Beheneth-25 Methacrylate Crosspolymer); the AMPS Na/vinylpyrrolidone copolymers, such as that sold under the name Aristoflex AVS (INCI: Sodium Acryloyldimethyltaurate/VP Copolymer); AMPS NH4/2-Carboxyethylacrylate copolymers, such as the product sold under the name Aristoflex TAC (INCI: Ammonium Acryloyldimethyltaurate/carboxyethyl crosspolymer); AMPS Na/Acrylic Acid/Sodium Acrylate/Dimethylacrylamide copolymers, such as the product sold under the name Simulgel SMS88 (INCI: Sodium Acrylate/Acryloyldimethyltaurate/Dimethylacrylamide Crosspolymer & Isohexadecane & Polysorbate 60); the AMPS Na/Sodium Acrylate copolymers such as those sold under the names Simulgel EG (INCI: Sodium Acrylate/Sodium Acryloyldimethyl Taurate Copolymer & Isohexadecane & Polysorbate 80) or Simulgel EPG (INCI: Sodium Acrylate/Sodium Acryloyldimethyl Taurate Copolymer & Polyisobutene & Caprylyl/Capryl Glucoside); AMPS Na/Acrylamide copolymers such as those sold under the names Simulgel 600 (INCI: Acrylamide/Sodium Acryloyldimethyltaurate/Isohexadecane/Polysorbate-80) or Sepigel 305 (INCI: Polyacrylamide/C13-C14 Isoparaffin/Laureth-7); Methyl Meth-acrylate Crosspolymer, such as those sold under the names SEPIMAT™ SB by the company SEPPIC; the AMPS Na/hydroxyethyl acrylate copolymers, such as those sold under the names Simulgel NS (INCI: hydroxyethyl acrylate/sodium acryloyldimethyltaurate copolymer & squalane & polysorbate-60), Simulgel INS 100 (INCI: Hydroxyethyl Acrylate/Sodium Acryloyldimethyl Taurate Copolymer & Isohexadecane & Polysorbate 60), Simulgel FL (INCI: Hydroxyethyl Acrylate/Sodium Acryloyldimethyl Taurate Copolymer & Isohexadecane & Polysorbate 60), Sepinov WEO or Sepinov EMT (INCI: hydroxyethyl acrylate/sodium acryloyldimethyltaurate copolymer); acryloyl Dimethyltaurate/Sodium Acrylate/Dimethylacrylamide crosspolymers such as that sold under the name Sepinov P88 (INCI: Sodium Acrylate/Acryloyldimethyl-taurate/Dimethylacrylamide Crosspolymer); and their mixtures.

Among the synthetic texturing agents, mention may also be made of PVP such as that sold under the name FlexiThix polymer or Nylon-6, such as that sold under the name Orgasol® 1002 D NAT COS.

Among the synthetic texturizing agents, the amphiphilic and anionic associative polymers are especially represented by the acrylates/Steareth-20 Methacrylate Copolymer such as that sold under the name Aculyn 22; acrylates/Beheneth-25 Methacrylate Copolymer such as that sold under the name Aculyn 28; C30-38 Olefin/Isopropyl Maleate/MA Copolymer such as that sold under the name Performa V 1608; Acrylates/Steareth-20 Methacrylate Crosspolymer such as that sold under the name Aculyn 88; Polyacrylate Crosspolymer-6 such as that sold under the name Sepimax Zen; Acrylates/C10-C30 Alkyl Acrylate Crosspolymers such as those mentioned above; and their mixtures.

Among the synthetic texturizing agents, the amphiphilic and nonionic associative polymers are especially represented by the PEG-150 distearate, such as that sold under the name Emanon 3299V; PEG-150/Decyl Alcohol/SMDI Copolymer such as that sold under the name Aculyn 44; PEG-150/stearyl alcohol/SMDI copolymer such as that sold under the name Aculyn 46; acrylates/ceteth-20 itaconate copolymer such as that sold under the name Structure 3001 by AkzoNobel Personal Care; polyurethane polyethers such as those sold under the names Rheolate FX 1100, Rheolate 205, Rheolate 208/204/212, Elfacos T1212, Acrysol RM 184/RM 2020, Adeka Nol GT-700/GT-730; polyurethane-39 such as that sold under the name Luvigel Star; cetyl hydroxyethylcellulose such as those sold under the names Natrosol™ Plus or PolySurf™ 67; and their mixtures.

As texturizing agents of the aqueous phase, mention may also be made of clays, in particular represented by bentonite such as that sold under the names Veegum, Veegum HS or Vanatural; montmorillonite, hectorite such as that marketed under the names Bentone series or Hectone series; kaolinite, and their mixtures.

Preferably, the aqueous phase, in particular continuous phase, may comprise as texturizing agent at least one crosslinked copolymer as described below, and in particular Carbopol® Aqua SF1 (INCI name=Acrylates copolymer).

The texturizing agents that may be used according to the invention may be organic or inorganic, polymeric or molecular lipophilic texturing agents.

As a mineral lipophilic texturizing agent, mention may be made of optionally modified clays such as hectorites modified with a C₁₀-C₂₂ ammonium chloride, such as hectorite modified with di-stearyl-dimethyl ammonium chloride such as, for example that marketed under the name Bentone 38V® by the company ELEMENTIS.

Mention may also be made of fumed silica optionally treated with a hydrophobic surface whose particle size is less than 1 μm. It is in fact possible to chemically modify the surface of the silica by chemical reaction to generate a decrease in the number of silanol groups present on the surface of the silica. In particular, it is possible to substitute silanol groups with hydrophobic groups: a hydrophobic silica is then obtained.

The hydrophobic groups may be:
trimethylsiloxyl groups that are especially obtained by treating fumed silica in the presence of hexa-methyldisilazane. Silicas thus treated are called “silica silylate” according to the CTFA (8th edition, 2000). They are for example sold under the names Aerosil R812® by the company DEGUSSA, CAB-O-SIL TS-530® by CABOT; or
dimethylsilyloxyl or polydimethylsiloxane groups, which are especially obtained by treating fumed silica in the presence of polydimethylsiloxane or dimethyldichlorosilane. Silicas thus treated are called “silica dimethyl silylate” according to the CTFA (8th edition, 2000). They are for example sold under the names Aerosil R972®, and Aerosil R974® by the company DEGUSSA, CAB-O-SIL TS-610® and CAB-O-SIL TS-720® by CABOT.

The hydrophobic fumed silica has, in particular, a particle size that may be nanometric to micrometric, for example ranging from about 5 to 200 nm.

The polymeric organic lipophilic texturizing agents are, for example, partially or fully crosslinked elastomeric organopolysiloxanes of three-dimensional structure, such as those sold under the names KSG6®, KSG16® and KSG18® by the company SHIN-ETSU, by Trefil E-505C® and Trefil E-506C® by DOW-CORNING, Gransil SR-CYC®, SR DMF10®, SR-DC556®, SR SCYC Gel®, SR DMF 10 Gel® and SR DC 556 Gel® by the company GRANT INDUSTRIES, SF 1204® and JK 113® by the company GENERAL ELECTRIC; ethylcellulose such as that sold under the name Ethocel® by the company DOW CHEMICAL; galactomannans having from one to six, and in particular from two to four, hydroxyl groups per sac, substituted by a saturated or unsaturated alkyl chain, such as guar gum alkylated by C₁-C₆ alkyl chains, and, in particular, C₁-C₃ and mixtures thereof. Block copolymers of the “diblock”, “triblock” or “radial” type of the polystyrene/polyisoprene, polystyrene/polybutadiene type, such as those sold under the name Luvitol HSB® by the company BASF, of the polystyrene/copoly (ethylene-propylene) type such as those sold under the name Kraton® by Shell Chemical Co. or else from the polystyrene/copoly (ethylene-butylene) type, mixtures of triblock and radial (star) copolymers in isododecane, such as those marketed by the PENRECO company under the name Versagel® such as the mixture of butylene/ethylene/styrene triblock copolymer and star copolymer ethylene/propylene/tyrene in isododecane (Versagel M 5960).

Mention may also be made, as a lipophilic texturizing agent, of polymers having a weight average molecular weight of less than 100,000, comprising a) a polymeric backbone having hydrocarbon-based repeating units provided with at least one heteroatom, and, optionally, b) at least one pendant fatty chain and/or at least one optionally functionalized terminal fatty chain having from 6 to 120 carbon atoms and being bonded to these hydrocarbon units, as described in applications WO 02/056847 and WO 02/47619, in particular the polyamide resins (especially comprising alkyl groups having 12 to 22 carbon atoms) such as those described in U.S. Pat. No. 5,783,657.

As an example of a polyamide resin that may be used according to the present invention, mention may be made of Uniclear 100 VG® marketed by ARIZONA CHEMICAL.

Among the lipophilic texture agents that may be used in the emulsions according to the invention, mention may also be made of dextrin and fatty acid esters, such as dextrin palmitates, in particular such as those sold under the names Rheopearl TL® or Rheopearl KL® by the company CHIBA FLOUR.

It is also possible to use polyorganosiloxane type silicone polyamides such as those described in U.S. Pat. No. 5,874,069, U.S. Pat. No. 5,919,441, U.S. Pat. No. 6,051,216 and U.S. Pat. No. 5,981,680.

These silicone polymers may belong to the following two families:
polyorganosiloxanes comprising at least two groups capable of establishing hydrogen interactions, wherein these two groups are located in the polymer chain, and/or polyorganosiloxanes comprising at least two groups capable of establishing hydrogen interactions, wherein these two groups are located on grafts or branches.

Additional Compound(s)

The emulsions of the invention may further comprise powders, flakes, dyes, preservatives, humectants, stabilizers, chelators, emollients, etc., or any of the usual cosmetic additives, and mixtures thereof.

The emulsions according to the invention may furthermore comprise at least one active agent, preferably chosen from among moisturizing agents, healing agents, depigmenting agents, UV-screening agents, desquamating agents, antioxidants, active agents stimulating the synthesis of macromolecular dermal and/or epidermal, dermo-decontracting agents, antiperspirants, soothing agents, anti-aging agents and mixtures thereof.

Of course, persons skilled in the art will take care to choose the additional compound(s) and/or their quantity as a function of the aqueous or fatty nature of the phase in question and/or in such a way that (i) the advantageous properties of an emulsion according to the invention and (ii) the integrity of the drops forming the emulsion, are not, or not substantially, impaired by the addition envisaged. These adjustments are within the competence of the person skilled in the art.

In particular, and as is apparent from Example 3 below, the use of an antioxidant makes it possible to effectively reduce or prevent the phenomenon of yellowing of an emulsion present in a packaging device according to the invention which, for obvious reasons, is undesirable. This thus leads to further strengthening the stability of an emulsion according to the invention present in a packaging device according to the invention.

An emulsion according to the invention thus advantageously comprises an effective amount of antioxidant(s), in particular an amount sufficient to reduce, or even prevent, the yellowing phenomenon of an emulsion according to the invention present in a packaging device according to the invention.

Advantageously, an emulsion according to the invention comprises an antioxidant content:
greater than 0% by weight relative to the total weight of the emulsion, and
less than or equal to 0.04% by weight relative to the total weight of the emulsion, and, in particular, between 0.002% and 0.03%, preferably between 0.004% and 0.02%, by weight relative to the total weight of the emulsion.

Advantageously, an emulsion according to the invention comprises a weight ratio “antioxidant(s)/perfume agent(s)” of between 2.8×10⁻⁵ and 0.02, preferably between 5.7×10⁻⁵ and 0.015, in particular between 1.10⁻⁴ and 0.01, and more preferably between 2.10⁻⁴ and 0.008.

According to one embodiment, the emulsions of the invention, in particular in the case of an oil-in-water type emulsion, comprise glycerine. Preferably, the emulsions of the invention comprise at least 5% by weight of glycerine relative to the total weight of the emulsions. In fact, in addition to the texture, the emulsions according to the invention provide another advantage over “conventional” emulsions because they allow the use of glycerin, moreover at high levels.

They may in particular comprise glycerin with a content greater than or equal to 10%, greater than or equal to 20%, greater than or equal to 30%, greater than or equal to 40%, or even up to 50%, by weight, relative to the total weight of the emulsion.

For obvious reasons, glycerin is present in the aqueous phase of an emulsion according to the invention.

In the case of emulsions comprising a high content of glycerine, the inventors have been able to advantageously observe an absence of sticky effect of the emulsion during the application of the emulsion by means of the device according to the invention.

Preparation Method

The emulsions according to the invention have the advantage of being able to be prepared according to a simple “non-microfluidic” method, i.e. by simple emulsification. As in a conventional emulsion, an aqueous solution (or phase) and a solution (or phase) fatty (or oily) are prepared separately. It is the stirring addition of the “fatty” phase in the aqueous phase that creates a direct emulsion.

The aqueous and fatty phases are thus prepared prior to their contacting one another to form an emulsion according to the invention. During the preparation of the aqueous phase and/or the fatty phase, the presence of certain raw materials may require adjustments in the method to prepare the aqueous phase and/or the fatty phase.

In particular, a heating step (between 40° C. and 150° C., in particular between 50° C. and 90° C.) may be necessary in the preparation of the fatty phase in order to solubilize the solid fatty substance(s) at ambient temperature and pressure and/or the antioxidant(s) may be incorporated.

Equally, the addition of the perfume(s) in the fatty phase is advantageously carried out at room temperature to prevent any degradation of its olfactory note.

Moreover, the presence, in the fatty phase, of solid fatty substance(s) at ambient temperature and pressure, as envisaged above, may necessitate adjustments in the method for preparing an emulsion according to the invention. In particular, the method for preparing such an emulsion according to the invention may comprise a heating step (between 40° C. and 150° C., in particular between 50° C. and 90° C.) of the fatty phase before mixing/bringing into contact the fatty phase with the aqueous phase, or even maintaining this heating during mixing of the fatty phase with the aqueous phase, and advantageously until the desired emulsion is obtained.

These adjustments are within the general competence of persons skilled in the art.

In the case of an oil-in-water emulsion, the solutions (or fluids) used to constitute the continuous aqueous phase and the dispersed fatty phase are respectively designated External Fluid (FE) and Internal Fluid (FI).

In view of the foregoing, the FI fluid comprises at least one first precursor polymer of the coacervate, in particular a cationic polymer, and in particular amodimethicone and, optionally, at least one oil, at least one solid fatty substance at ambient temperature and pressure, in particular as defined above, at least one perfuming agent and/or at least one additional compound mentioned above.

The FE fluid comprises at least water and at least a second precursor polymer of the coacervate that is different from the first precursor polymer of the coacervate, in particular an anionic polymer, and, in particular, the carbomer, and optionally, at least one crosslinked polymer/copolymer, at least one perfuming agent and/or at least one additional compound as mentioned above, or even a base, preservatives and/or other water-soluble products such as glycerine.

According to one embodiment, the method for preparing an emulsion according to the invention of the oil-in-water type comprises a drop-forming step comprising:
bringing into contact an FE fluid and an FI fluid as defined above; and
the formation of drops of fatty phase, consisting of the fluid F1, dispersed in a continuous aqueous phase consisting of fluid FE, wherein the drops comprise a shell insulating the heart of the droplets of the fatty phase of the dispersion.

According to one embodiment, the drop formation step may further comprise a step of injecting a solution to increase the viscosity of the continuous aqueous phase of the FE fluid. Preferably, the solution increasing the viscosity is aqueous. This solution for increasing the viscosity is typically injected into the aqueous FE fluid after formation of the dispersion according to the invention, and thus after formation of the drops.

According to one embodiment, the solution for increasing the viscosity comprises a base, in particular an alkaline hydroxide, such as sodium hydroxide.

According to one embodiment, in particular when the FI fluid comprises at least one solid fat at ambient temperature and pressure and/or at least one antioxidant as described above, the method for preparing an emulsion according to the invention may furthermore comprising a step of heating the F1 fluid to a temperature of from 40° C. to 150° C., preferably from 50° C. to 90° C., prior to the above-mentioned step of forming the drops, and therefore before mixing/bringing in contact of the fatty phase with the continuous aqueous phase. Advantageously, the method for preparing an emulsion according to the invention comprising such an FI fluid is such that the steps of bringing into contact an FE fluid and an FI fluid and forming drops of the fatty phase, consisting of the FI fluid, dispersed in a continuous aqueous phase consisting of FE fluid, is carried out at a temperature of 40° C. to 150° C., preferably 50° C. to 90° C. In other words, such a method comprises maintaining the aforementioned heating during the mixing of the fatty phase with the aqueous phase, and advantageously until the desired emulsion is obtained.

According to one embodiment, the method of preparation may further comprise, between the heating step and the step of forming the drops, a step of lowering the temperature of the fluid FI, if necessary to room temperature.

Uses

Preferably, the emulsion according to the invention is usable immediately at the end of the above-mentioned preparation method, as a composition, in particular as a cosmetic composition.

An emulsion according to the invention may also be in the form of a composition, in particular a cosmetic composition, and more particularly a perfume composition.

Such a composition, in particular a cosmetic composition, comprises at least one emulsion according to the invention in combination with a physiologically acceptable medium.

By “physiologically acceptable medium” is meant a medium that is particularly suitable for applying a composition of the invention to keratin materials.

According to one embodiment, the composition is a cosmetic composition.

The term “keratin materials” is intended to denote in particular the skin, the lips, the nails, the eyelashes or the eyebrows, preferably the skin, and especially the skin of the neck. The physiologically acceptable medium is generally adapted to the nature of the support to which the composition is to be applied, as well as to the appearance under which the composition is to be packaged.

According to one embodiment, the physiologically acceptable medium is directly represented by the aqueous continuous phase as described above.

According to one embodiment, the emulsions or cosmetic compositions according to the invention are used for makeup and/or the care of keratin materials and/or for perfuming a keratinous material, in particular the skin.

Preferably, a composition according to the invention does not comprise a surfactant.

The cosmetic compositions according to the invention may be perfume (or a perfuming product) for the care, sun protection, cleaning (makeup removal), hygiene or makeup of keratin materials, including the skin.

According to one embodiment, the emulsions or compositions of the invention are in the form of a foundation, a makeup remover, a facial and/or body and/or hair care, an anti-aging care, sun protection, oily skin care, whitening care, moisturizer, BB cream, tinted cream or foundation, facial and/or or body cleanser, a shower gel or a shampoo.

According to one embodiment, the emulsions or compositions of the invention are not tinted creams or foundations.

A care composition according to the invention may be, in particular, a solar composition, a care cream, a serum or a deodorant.

Preferably, a composition according to the invention, when it comprises at least one perfuming agent, as indicated above, is a perfuming composition (or perfume). In other words, such a composition according to the invention is a perfume which differs from “conventional” perfumes by its galenic and its mode of application.

According to one embodiment in which the emulsion according to the invention comprises at least one perfuming agent and glycerol, which, moreover, is in a content greater than or equal to 5% by weight relative to the total weight of the emulsion, or even a composition comprising the emulsion, the emulsion or composition is particularly advantageous in that it may be used as a perfuming and caring composition for a keratin material, in particular the skin. This embodiment is particularly interesting because it is not obvious that the moisturizing nature of such a composition does not alter the desired olfactory effect, and vice versa.

The emulsions or compositions according to the invention may be in various forms, in particular in the form of cream, balm, lotion, serum, gel, gel cream or mist.

The present invention also relates to a non-therapeutic cosmetic method for makeup and/or care of a keratin material and/or for improving perfume retention on a keratinous material, comprising the following steps:
i) bringing the application means into contact with an emulsion of the device as described above; and
ii) applying the emulsion, present on the means of application, to a keratin material, preferably the skin.

According to one embodiment, the bringing into contact of the applicator with the emulsion is carried out by pressure of the applicator, in particular by the user, on the element having at least one orifice of the receptacle.

In particular, when the element is a sponge, the emulsion is transferred from the sponge to the applicator.

In particular, when the element is a grid or sieve, the emulsion is transferred through the holes (grid) or mesh (sieve) to the applicator.

Throughout the description, including the claims, the phrase “comprising one” should be understood as being synonymous with “comprising at least one”, unless the opposite is specified.

The expressions “comprised between . . . and . . . ”, “comprised from . . . to . . . ” and “from . . . to . . . ” must be understood as being inclusive, unless otherwise specified.

The amounts of the ingredients in the examples are expressed as percentage by weight relative to the total weight of the composition, unless otherwise indicated.

### EXAMPLES

### Example 1 Preparation of a “Cream Cushion” with a Fragrance Composition of Oil-in-Water Emulsion Type

The composition of Example 1 consists of the following:

| Name | INCI name | % w/w |
|---|---|---|
| Osmosis water | Aqua | 82.48% |
| Perfume | Fragrance | 10% |
| DUB ININ Grade A | Isononyl Isononanoate | 1% |
| CAS3131 PILOT | Amodimethicone | 0.02% |
| Carbopol 981 | Carbomer | 0.18% |
| Crosspolymer | Acrylates crosspolymer-4 | 2.70% |
| Carbopol Aqua SF2 | | |
| Microcare ® PE | Phenoxyethanol | 0.72% |
| Microcare ® emollient | Pentylene glycol | 1.80% |
| PTG | | |
| Hepes | Hydroxyethylpiperazine | 0.90% |
| | Ethane Sulfonic Acid Hydro | |
| Edeta ® BD | Disodium EDTA | 0.01% |
| NaOH | Sodium Hydroxyde | 0.19% |

| | |
|---|---|
| Total | 100.00% |

The composition of Example 1 is prepared according to the following protocol:

Preparation of the Aqueous Phase

Half of the final water is mixed with the preservatives. It is stirred with a deflocculating type blade for 15 minutes at 300 rpm.

Stirring is stopped to incorporate the carbomer, the hydration is left for 1 hour and then stirred with a deflocculator quickly but without incorporating air bubbles, for 2 hours.

The crosspolymer is added and stirred until completely dispersed.

A solution is prepared separately with the Hepes, the sodium hydroxide and the remainder of the water, which is then added slowly to the preceding solution, still stirring.

Preparation of the Fatty Phase (Perfuming Phase):

Separately, the perfuming phase is prepared by mixing isononyl isononanoate, amodimethicone and perfume.

Preparation of the Emulsion:

The perfuming phase is then slowly incorporated into the aqueous phase with gentle stirring until complete dispersion.

Alternatively, the solution with Hepes, sodium hydroxide and the remainder of water may be added after dispersion of the perfuming phase in the carbomer solution.

This composition is then placed in a packaging device according to the invention (i.e. of the “cream cushion” type); in the receptacle of the device, wherein the composition is soaked/impregnated within a cellular sponge (i.e. the element having at least one orifice).

Protocol for Application on a Keratin Material

With the aid of a puff (i.e. the means of application), the user exerts pressure on the sponge comprising the above composition and thus takes up a desired amount of the composition, without contact between the hand of the user and the composition.

In addition, the user may easily adjust the amount of composition taken up by exerting more or less pressure on the sponge. In other words, the puff may absorb the right dose of the composition.

Next, the user brings the puff coated at least partially with the composition into contact with the skin of the neck.

Results

Initially, in view of the cosmetic purpose considered, namely perfuming, the user may be surprised at the mode of packaging and the galenic composition.

Then, upon applying the above composition to the skin by using the device according to the invention, the user discovers the following advantages:
a feeling of freshness on the skin,
the supply of a light product,
easy application: the puff used to apply the product is soft, pleasant and easy to use,
the composition is multi-function, or even personalized as the puff can absorb the right dose of the composition so that the application of the latter may vary on the levels of coverage and/or loading of the perfume, from the most subtle to the most intense,
the sponge is rechargeable, which is environmentally friendly and allows adjustment of the color according to the season,
there is no contact between the fingers and the composition, and
the product is “nomadic” because it is small and easy to carry.

### Example 2: Comparative Test

A comparison is made of the composition described in Example 1 above with respect to the mode of application.

Each user considered for this comparison performs an application on the skin of the composition above via two different modes of application, i.e.:
the first mode of application, on the left part of the neck, corresponds to a “conventional” application of the spray type, and
the second mode of application, on the right side of the neck, corresponds to an application using the device according to the invention.

The users' impressions, particularly in terms of the persistence of the perfume and the intensity of the latter, are then collected.

Results

In addition to the aforementioned advantages, the composition applied to the skin using the device according to the invention offers:
a better persistence of the olfactory effect conferred by the perfuming agent present in the composition, and
improved olfactory power.

### Example 3: Comparative Test on the Yellowing of Oil-in-Water Emulsion Perfuming Compositions

| | | Final composition | | | | | |
|---|---|---|---|---|---|---|---|
| | | A | B | C | D | E | F |
| Name | INCI name | final % w/w | | | | | |
| AQUEOUS | sub-total | 92.00 | | | | | |
| PHASE GEL | | | | | | | |
| Osmosis water | Aqua | 73.45 | | | | | |
| Microcare PE | Phenoxyethanol, aqua | 0.8 | | | | | |
| Microcare | Pentylene glycol, aqua | 2.00 | | | | | |
| emollient PTG | | | | | | | |
| Glycerine codex | Glycerin, aqua | 8 | | | | | |
| (99%) | | | | | | | |
| Zemea | Propanediol, aqua | 2 | | | | | |
| propanediol | | | | | | | |
| Butylene glycol | Butylene glycol, aqua | 1 | | | | | |
| 1,3 | | | | | | | |
| EDETA bd | Disodium EDTA | 0.03 | | | | | |
| Carbopol ultrez | Carbomer | 0.4 | | | | | |
| 10 polymer | | | | | | | |
| Carbopol aqua | Aqua, Acrylates | 2.00 | | | | | |
| SF-1 polymer | copolymer | | | | | | |
| Hepes-luv | Hydroxyethylpiperazine | 0.92 | | | | | |
| | ethane sulfonic acid, | | | | | | |
| | Aqua | | | | | | |
| Sepimat sb | Methyl methacrylate | 0.5 | | | | | |
| | crosspolymer, Aqua | | | | | | |
| Orgasol 1002 | Nylon-6, Aqua | 0.5 | | | | | |
| dnat cos | | | | | | | |
| Sodium | Sodium hydroxide | 0.40 | | | | | |
| hydroxide pellets | | | | | | | |
| prs codex | | | | | | | |
| FATTY PHASE | sub-total | 8.00 | 8.00 | 8.00 | 8.00 | 8.00 | 8.00 |
| DUB ININ | lsononyl isononanoate, | 4.98 | 4.96 | 4.94 | 4.92 | 4.88 | 4.976 |
| Grade A | Aqua | | | | | | |
| TINOGARD TT | Pentaerythrityl tetra-di-t-butyl | 0.00 | 0.02 | 0.04 | 0.06 | 0.10 | 0.004 |
| | hydroxyhydrocinnamate | | | | | | |
| Perfume | Fragance | 3.00 | 3.00 | 3.00 | 3.00 | 3.00 | 3.00 |
| CAS-3131 | Amodimethicone | 0.02 | 0.02 | 0.02 | 0.02 | 0.02 | 0.02 |
| PILOT | | | | | | | |

| | | | | | | |
|---|---|---|---|---|---|---|
| TOTAL | 100.00 | 100.00 | 100.00 | 100.00 | 100.00 | 100.00 |

The compositions A to F of Example 3 comprise the following ingredients:

The preparation protocol for composition A is similar to that described in Example 1.

The protocol for the preparation of compositions B to F is similar to that described in Example 1, with the difference that the preparation of the fatty phase (or perfuming phase) comprises a heating step (approximately 45° C.) before adding the Tinogard TT in DUB ININ to solubilize this antioxidant satisfactorily.

These compositions A to F are then each placed in a packaging device according to the invention (i.e. of the “cream cushion” type).

With a composition A (i.e. according to the invention but devoid of antioxidant), the inventors observed the appearance of a slight yellowing of the latter 15 days after its manufacture.

The addition of an antioxidant (i.e. TINOGARD TT) was therefore envisaged in different concentrations (see compositions B to F).

Stability tests, especially in the yellowing, of compositions A to F were carried out for 1 month, at room temperature (RT) and at 45° C.

The yellowing is identified on the basis of a visual observation and the intensity of this yellowing is identified according to the following scale:

| Value | Yellowing intensity of the composition |
|---|---|
| 0 | No yellowing (white composition) |
| 1 | Light |
| 2 | Medium |
| 3 | High |
| 4 | Very high |

A visual observation of compositions A to F at TA and 45° C. was performed once a week.

Results:

| Time | A | | B | | C | | D | | E | | F | |
|---|---|---|---|---|---|---|---|---|---|---|---|---|
| (in weeks) | TA | 45° C. | TA | 45° C. | TA | 45° C. | TA | 45° C. | TA | 45° C. | TA | 45° C. |
| 0 | 0 | 0 | 0 | 0 | 0 | 0 | 0 | 0 | 0 | 0 | 0 | 0 |
| 1 | 0 | 0 | 0 | 0 | 0 | 0 | 0 | 0 | 0 | 0 | 0 | 0 |
| 2 | 1 | 1 | 0 | 0 | 1 | 1 | 1 | 1 | 1 | 2 | 0 | 0 |
| 3 | 2 | 2 | 1 | 1 | 2 | 3 | 2 | 3 | 2 | 3 | 1 | 1 |
| 4 | 2 | 3 | 1 | 1 | 3 | 4 | 3 | 4 | 4 | 4 | 1 | 1 |

A yellowing of composition A (no antioxidant) was observed from 2 weeks after manufacture of the composition A.

A slight yellowing is observed towards the end of the investigation for compositions B and F.

In contrast, a higher intensity yellowing of compositions C to E was observed from 2 weeks after manufacture of the compositions C to E, wherein the intensity of this yellowing is proportional to the concentration of antioxidant.

Conclusion:

The use of an antioxidant makes it possible to reduce the phenomenon of yellowing of an emulsion according to the invention in a particular concentration range, i.e.:
greater than 0% by weight relative to the total weight of the emulsion, and
less than 0.04% by weight relative to the total weight of the emulsion.

In fact, beyond 0.04% by weight of antioxidant relative to the total weight of the composition, TINOGARD TT has an undesirable prooxidant action.

### Example 4: Preparation of a Cream Cushion with an Oil-in-Water Emulsion Perfuming Composition Obtained Via a Hot Method

The composition of Example 4 consists of the following ingredients:

| | | | Phases | | |
|---|---|---|---|---|---|
| Raw material | Supplier | INCI name | % w/w | Final % w/w | Phase |
| AQUEOUS PHASE | | Sub-total | 100.00 | 83.467 | |
| Osmosis water | / | Aqua | 76.34 | 63.72 | A |
| Glycerine codex | Interchimie | Glycerin. Aqua | 5.99 | 5.00 | A |
| Butylene glycol | Interchimie | Butylene glycol | 5.99 | 5.00 | A |
| Zemea propanediol | Dupont Tate | Propanediol. Aqua | 5.99 | 5.00 | A |
| | & Lyle | | | | |
| EDETA bd | BASF | Disodium EDTA | 0.04 | 0.03 | A |
| Carbopol ultrez 30 polymer | Lubrizol | Carbomer | 0.12 | 0.10 | B |
| Sepinov emt 10 | Seppic | Hydroxyethyl acrylate/ | 0.96 | 0.80 | C |
| | | sodium acryloyldimethyl | | | |
| | | taurate copolymer | | | |
| Microcare PE | Thor | Phenoxyethanol. Aqua | 0.96 | 0.80 | A |
| Microcare emollient PTG | Thor | Pentylene glycol. Aqua | 2.40 | 2.00 | A |
| Orgasol caresse | Arkema | Polyamide-5 | 1.20 | 1.00 | D |
| Sodium hydroxide pellets prs | Panréac | Sodium hydroxide | 0.02 | 0.017 | |
| codex | | | | | |
| FATTY PHASE | | Sub-total | 100.00 | 16.53 | |
| CAS-3131 PILOT | Nusil | Amodimethicone | 0.200 | 0.033 | E′ |
| CSF-3100 (50 cSt) | Nusil | Dimethicone | 12.10 | 2.00 | E |
| Lanette O OR | Basf | Cetearyl alcohol | 6.05 | 1.00 | E |
| Lipex shea | Unipex | Butyrospermum parkii butter | 18.15 | 3.00 | E |
| Refined meadowfoam oil | Biocosmethic | Limnanthes alba seed oil | 12.10 | 2.00 | E |
| Crodamol ml-lq-(rb) | Croda | Myristyl lactate | 6.05 | 1.00 | E |
| Cutina pes | Basf | Pentaerythrityl distearate | 9.07 | 1.50 | E |
| Crodamol ptis | Croda | Pentaerythrityl tetraisostearate | 18.15 | 3.00 | E |
| Perfume | / | Fragrance | 18.15 | 3.00 | F |
| TOTAL | | | | 100.00 | |
| Soda solition production 10% | | Sodium Hydroxyde | 0.20 | 0.167 | G |

The composition of Example 4 is prepared according to the following protocol:

Preparation of the Aqueous Phase:

In a first receptacle and at 60° C., add while stirring the raw materials A, then add gradually while stirring the raw materials B, C and D.

Preparation of the Fatty Phase (Perfuming Phase):

In a second receptacle and at 60° C., add while stirring the raw materials E, then add and homogenize the raw material E′ (still at 60° C.).

Preparation of the Emulsion:

At 60° C., the perfuming phase is incorporated in the aqueous phase with vigorous stirring for 15 minutes.

Then, the mixture is cooled to 25° C. and then the perfume (F) is added.

Finally, the emulsion is neutralized with the 10% sodium hydroxide solution (G) and stirred until a homogeneous mixture is obtained.

This composition is then placed in a packaging device according to the invention (i.e. of the “cream cushion” type); in the receptacle of the device, wherein the composition is soaked/impregnated within a cellular sponge (i.e. the element having at least one orifice).

The protocol for application on a keratin material and the results are identical to those described in Example 1.

1. Packaging device comprising at least:
i) a receptacle comprising at least one reservoir containing at least one emulsion comprising a continuous phase and a dispersed phase in the form of drops, wherein the drops comprise a shell formed by at least one cationic polymer and at least one anionic polymer;
ii) means for applying the emulsion to a keratin material; and
iii) an element having at least one orifice allowing the passage of the emulsion from the reservoir to the application means.

2. The device according to claim 1, wherein the element comprising at least one orifice is selected from the group consisting of a sponge, a rigid plate pierced with at least one hole, a net and a sieve.

3. The device according to claim 2, wherein the element comprising at least one orifice is a cellular sponge.

4. The device of claim 1, wherein the application means is selected from the group consisting of a brush, a puff, a pad, a foam, and a mini-brush.

5. The device of claim 1, wherein the emulsion is an oil-in-water emulsion.

6. The device of claim 1, wherein the cationic polymer is a silicone polymer modified with at least one primary, secondary or tertiary amine function.

7. The device of claim 1, wherein each drop comprises from 0.01% to 10% by weight of cationic polymer(s), relative to the total weight of the phase comprising the cationic polymer(s).

8. The device of claim 1, wherein the anionic polymer is a carbomer or a crosslinked copolymer acrylates/C₁₀₋₃₀ alkyl acrylate.

9. The device of claim 1, wherein the emulsion comprises from 0.01% to 5% by weight of anionic polymer(s), relative to the total weight of the emulsion.

10. The device of claim 1, wherein the emulsion comprises at least 2% by weight of perfuming agent(s) relative to the total weight of the emulsion.

11. The device of claim 1, wherein the emulsion further comprises at least one crosslinked polymer or at least one crosslinked copolymer, wherein the crosslinked polymer or crosslinked copolymer comprises at least one unit derived from the polymerization of at least one of the monomers selected from the group consisting of acrylic acid and/or methacrylic acid and/or alkyl acrylate with 1 to 30 carbon atoms, or their salts.

12. The device according to claim 11, wherein the emulsion comprises from 0.1% to 10% by weight of crosslinked polymer or crosslinked copolymer(s) relative to the total weight of the emulsion.

13. The device of claim 1 wherein the emulsion comprises glycerin in a content greater than or equal to 5% by weight relative to the total weight of the emulsion.

14. The device of claim 1, wherein the emulsion further comprises at least one active agent selected from among hydrating agents, healing agents, depigmenting agents, UV filters, desquamating agents, antioxidant agents, active agents stimulating the synthesis of dermal and/or epidermal macromoleculars, dermo-decontracting agents, antiperspirants, soothing agents, anti-aging agents and their mixtures.

15. The device of claim 1, wherein the emulsion does not include surfactant.

16. The device of claim 1, wherein the emulsion is in the form of a composition.

17. A non-therapeutic cosmetic method for making up and/or caring for a keratin material and/or for improving perfume retention on a keratin material, comprising at least the following steps:
i) bringing the application means into contact with an emulsion of the device of claim 1; and
ii) applying the emulsion present on the application means to the keratin material.
